(19) United States
(12) Patent Application Publication
(10) Pub. No.: US 2018/0050171 A1
(43) Pub. Date: Feb. 22, 2018

(54) Closed Loop Scent Delivery System and Method of Use

(71) Applicant: INTERNATIONAL FLAVORS & FRAGRANCES INC., New York, NY (US)

(72) Inventors: Matthias Horst Tabert, Arverne, NY (US); Anshul Jain, East Brunswick, NJ (US)

(21) Appl. No.: 15/682,829

(22) Filed: Aug. 22, 2017

Related U.S. Application Data
(60) Provisional application No. 62/377,832, filed on Aug. 22, 2016.

(51) Int. Cl. A61M 21/02; C11D 3/50; A61B 5/00

(52) U.S. Cl. CPC A61M 21/02; C11D 3/50; A61M 2021/0016; A61B 5/0022; A61B 5/0004

(57) ABSTRACT

A system and method for combining fragrance compositions that have evidence-based benefits for modifying a physiological state of a subject are provided. The system is composed of a biometric readout device and environmental sensor module that provide physiological and environmental feedback to a digitally controlled scent delivery device, which is configured to delivery scent based upon physiological and environmental conditions.

This application claims the benefit of priority of U.S. Provisional Application No. 62/377,832, filed Aug. 22, 2016, the content of which is incorporated herein by reference in its entirety.

### INTRODUCTION

### Background

Proper memory function requires encoding of a memory during learning, consolidation of the memory in the hours and days that follow, and retrieval of the learned content during testing. Memory consolidation is the process whereby the brain transfers memories to long-term storage. Consolidation of memories occurs primarily during sleep. Deep, or ‘slow-wave’ sleep (SWS), is particularly important for consolidating long-term memories. Recent advances in the fields of neurobiology, psychology, and sleep research have characterized the important relationship between sleep and memory.

Sleep is required for normal memory consolidation and reduced sleep quality or quantity disrupts memory function. In people, memory and other higher cognitive functions can be improved by optimizing sleep quantity or sleep quality. Intensive training or learning causes an increase in the amount of SWS sleep during a subsequent night, suggesting that this phase of sleep is required for memories to be consolidated. In rodents, neurobiological studies have shown that patterns of activity among neurons in the hippocampus, a key brain region for memory, occur in a predictable and sequential pattern when a rodent is exploring a maze or other environment. The spatial memory represented by this experience is thought to be consolidated during sleep. Electrophysiological recordings during SWS have been used to identify ‘replay’ of the patterns of neural activity observed during previous experience, suggesting that replay is an important mechanism for consolidation of memories to long-term storage. Interruption of replay during sleep by electrical stimulation disrupts memory formation.

Normal cognitive function requires sufficient and well-structured sleep. Cognitive impairment due to sleep abnormalities occurs in healthy individuals that are sleep deprived and in patients with neurodevelopmental disorders such as Down syndrome, neurodegenerative disorders such as Alzheimer's disease, various forms of insomnia, sleep apnea, and other pathological conditions. Similarly, reduced memory function unrelated to disease occurs with normal aging, overnight shift work, drug or alcohol use, and other causes of sleep impairment or sleep disruption. For these various forms of cognitive dysfunction, strategies to alleviate or mitigate cognitive deficits with pharmaceutical, educational, and behavioral interventions have received significant attention but have not adequately addressed cognitive deficits.

Devices have been suggested for monitoring physiological responses such as sleep. See U.S. Pat. No. 8,157,730 and U.S. Pat. No. 8,961,415. Further, systems and methods for using scent to modify sleep and cognition have been suggested. See, U.S. Pat. No. 8,573,980 and WO 2017/119332. However, new methods for improving sleep patterns of healthy individuals or the lives of those with intellectual disabilities, age-related cognitive decline, and other forms of learning disability by improving memory and cognitive function are desired.

### SUMMARY OF THE INVENTION

This invention is a system that includes a biometric readout device, one or more environmental sensor modules, and a digitally controlled scent delivery device, wherein the biometric readout device and environmental sensor modules are in a closed feedback loop with the digitally controlled scent delivery device to control scent delivery parameters based on physiological and environmental conditions. In one embodiment, the biometric readout device includes at least one physiological sensor configured to detect or measure physiological information from a subject. In another embodiment, the environmental sensor modules include at least one environmental sensor configured to detect or measure environmental conditions such as temperature, sound, humidity, light or volatile organic chemicals in a vicinity of a subject. In a further embodiment, the system further includes one or more other sensory delivery devices including, but not limited to devices for modulating light, sound, temperature, pressure, visual stimulus or haptics. In a further embodiment, the system includes a digital controller, which receives data from the biometric readout device, the one or more environmental sensor modules and other data platforms.

A method for modulating a subject's physiological state is also provided. The method involves the steps of receiving physiological and environmental information from a subject via a biometric readout device and one or more environmental sensor modules associated with the subject; analyzing the received information to identify the physiological and/or environmental status associated with the subject; providing feedback to a scent delivery device based upon the subject's physiological and/or environmental status; and delivering a scent from the scent delivery device thereby modulating the subject's physiological state. In some embodiments, the method further includes the step of delivering one or more other sensory modalities (e.g., light, sound, temperature, pressure, visual stimulus and/or haptics) to provide a scent-based multisensory experience. In some embodiments, the subject's sleep cycle is modulated. In other embodiments, the performance of the subject during a cognitive or motor task (e.g., driving a car) is modulated.

### BRIEF DESCRIPTION OF THE DRAWINGS

FIG. 1 provides a schematic overview of the system 10 of the invention. VOC, volatile organic compounds; GSR, galvanic skin response; HR, heart rate; EMG, electromyography; and EEG, electroencephalography.

FIG. 2 depicts an example of a system of the invention including a biometric readout device 20, one or more environmental sensor modules 30, a digital controller 40, other data platforms 50, a scent delivery device 60 and one or more other optional sensory delivery devices 70, wherein the biometric readout device and environmental sensors are in a closed feedback loop with the digital controller, scent delivery device and other sensory delivery devices to control scent delivery and other sensory modality parameters based on a subject's 80 physiological and environmental conditions.

FIG. 3 shows skin conductance and scent profiles for a subject during sleep. The scent delivery device and galvanic skin response sensor were in a closed-loop and lavender scent was triggered when skin conductance (measure of stress) crossed a predetermined threshold.

### DETAILED DESCRIPTION OF THE INVENTION

The present invention provides a system and method for combining fragrance compositions, which have evidence-based benefits for modifying a physiological response in a subject, with digitally controlled scent coupled to other optional sensory display modules (ambient light, sound and haptics) and biometric readout devices, e.g., oPhone & fitbit, respectively, to create a digital, closed feedback loop, scent-based multisensory experience for consumers. In addition, the scent display device is connected or coupled to one or more environmental (e.g., VOC, temperature, humidity, lighting and sound etc.) sensors in parallel closed feedback loops to further control and modulate multisensory delivery parameters based upon environmental conditions.

The phrase “scent-based multisensory experience” refers to the integrated output of numerous sensory display modalities coupled to the scent-display in such a way as to modulate or modify the consumer experience for a specific use case, e.g., falling asleep, awakening from sleep, car driving, etc.

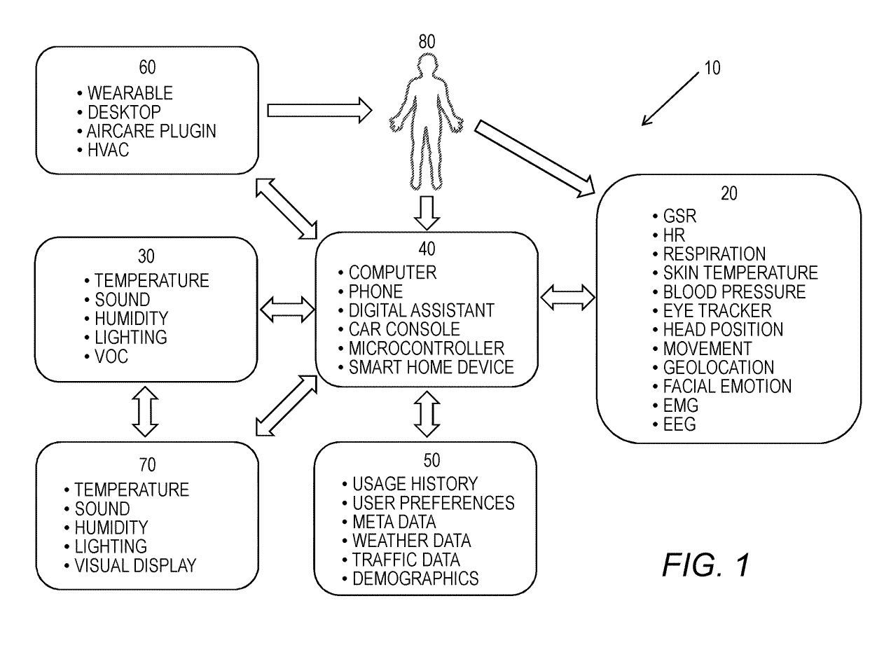

More specifically, the system 10 of the invention includes a biometric readout device 20; one or more environmental sensor modules 30; and a digital controller 40 for receiving, analyzing and sending data to and from the biometric readout device 20, the one or more environmental sensor modules 30, other data platforms 50, a scent display device 60, and optionally to one or more other sensory delivery devices 70. Based upon a subject's physiological and environmental conditions, system 10 functions in a closed feedback loop manner to control scent delivery and other sensory modality parameters to the subject (FIG. 1).

The phrase “other sensory modalities,” as used herein, refers to a stimulus, other than smell, that is capable of being perceived by a subject. “Other sensory modalities” include, but are not limited to, light, sound, temperature, pressure, or touch (i.e., haptic stimulation). When used in connection with scent delivery, the phrase “coupled to other sensory modalities” refers to scent delivery being directly or indirectly linked in real-time with the display or delivery of other sensory modalities to directly or indirectly modulate the multisensory experience of a subject based on sensor inputs and the physiological state of the subject thereby achieving a desired behavioral state (e.g., relaxed, calm, aroused, alert, energized etc.). In this respect, the phrase “other sensory delivery devices” refers to devices for modulating light (e.g., intensity and/or frequency), sound, temperature, pressure, visual stimulus and/or haptics.

The term “feedback” relates to measuring a subject's biometric/physiological signals such as blood pressure, heart rate, skin temperature, galvanic skin response (sweating), muscle tension, brain activity (EEG) etc., and environmental signals such as temperature, humidity, light, sound, and/or VOC levels and conveying such information to the scent delivery device and/or subject in real-time in order to provide the scent delivery device with data pertinent to the subject's physiological status and environment and/or raise the subject's awareness and conscious control of the related physiological activities. Herein, feedback is synonymous with personal physiological and environmental monitoring, where biochemical processes and environmental occurrences may be integrated into information for one or more individuals. For example, monitoring sleep patterns and air quality through the sensors described herein for the purpose of tracking, predicting, and/or controlling the sleep cycle is also considered feedback.

Devices for monitoring various physiological and environmental factors are connected or coupled to the scent delivery device and optionally one or more sensory delivery devices to provide biofeedback and environmental information to the scent delivery device and one or more optional sensory delivery devices. It will be understood that when an element is referred to as being “attached” to, “connected” to, “coupled” with, “contacting”, etc., another element, it can be directly attached to, connected to, or coupled with another element or may be indirectly attached to, connected to or coupled with another element by one or more intervening elements. By way of illustration, physiological and environmental sensors may be coupled to a scent delivery device via a digital controller. It will also be appreciated by those of skill in the art that the above-referenced terms include wired or wireless communication between the devices.

In some embodiments, the biometric readout device and environmental sensor module are in individual housings. In other embodiments, the biometric readout device and environmental sensor module are within the same housing. According to some embodiments of the present invention, real-time, noninvasive health and environmental monitors include a plurality of compact sensors integrated within one or more small, low-profile devices. Physiological and environmental data is collected and wirelessly transmitted, where the data can be stored and/or processed to provide information to the microprocessor of the scent delivery device.

The term “real-time” is used to describe a process of sensing, processing, or transmitting information in a time frame which is equal to or shorter than the minimum timescale at which the information is needed. For example, the real-time monitoring of pulse rate may result in a single average pulse-rate measurement every minute, averaged over 30 seconds. Typically, averaged physiological and environmental information is more relevant than instantaneous changes. Thus, in the context of the present invention, signals may sometimes be processed over several seconds, or even minutes, in order to generate a “real-time” response.

The term “monitoring” refers to the act of measuring, quantifying, qualifying, estimating, sensing, calculating, interpolating, extrapolating, inferring, deducing, or any combination of these actions. More generally, “monitoring” refers to a way of getting information via one or more sensing elements. For example, “blood health monitoring” includes monitoring blood gas levels, blood hydration, and metabolite/electrolyte levels.

The biometric readout device 20 and/or environmental sensor module 30 can take a variety of forms. For example, such monitors can be in the form of earpieces, bracelets, wristwatches, rings, garments, gloves, headbands, hats, wearable digital skin or patches. Preferably, the biometric readout device is an object worn on the skin. Since the hand has a special individual, intensive subcutaneous blood vessel pattern, in some embodiments, the monitor is a bracelet, wristwatch, ring or glove. Because the ear region is located next to a variety of “hot spots” for physiological an environmental sensing, including the tympanic membrane, the carotid artery, the paranasal sinus, etc., in some cases an earpiece monitor takes preference over other forms. Earpiece monitors can take the form of a hearing aid, an earplug, an entertaining speaker, the earpiece for an IPOD®, WALKMAN®, or other entertainment unit, a commercial headset for a phone operator, an earring, a gaming interface, or the like.

The biometric readout device and/or environmental sensor module of this invention can take advantage of commercially available open-architecture, ad hoc, wireless paradigms, such as BLUETOOTH®, Wi-Fi, or ZigBee and may be configured to transmit information wirelessly to the digital controller.

The biometric readout device and/or environmental sensor module may contain a plurality of sensors for monitoring personal health and environmental exposure. Health and environmental information, sensed by the sensors is transmitted wirelessly, in real-time, to the controller, which is capable of processing the data and provide feedback for scent delivery and optional other sensory delivery. In some embodiments, a user can monitor health and environmental exposure data in real-time, and may also access records of collected data throughout the day, week, month, etc., through an audio-visual display.

The term “physiological” refers to matter or energy of or from the body of a creature (e.g., humans, animals, etc.). In embodiments of the present invention, the term “physiological” is intended to be used broadly, covering both physical and psychological matter and energy of or from the body of an organism. However, in some cases, the term “psychological” is called-out separately to emphasize aspects of physiology that are more closely tied to conscious or subconscious brain activity rather than the activity of other organs, tissues, or cells. The term “physiological state” is used herein to refer to the physiological status of a subject or more particularly the physical, psychological, metabolic, emotional, mental, cognitive and/or pathophysiological status of the subject.

Each physiological sensor of the biometric readout device 20 is configured to detect and/or measure one or more of the following types of physiological information: heart rate, galvanic skin response or skin conductance response, pulse rate, breathing rate, blood flow, heartbeat signatures, cardio-pulmonary health, organ health, metabolism, electrolyte type and/or concentration, physical activity, caloric intake, caloric metabolism, blood metabolite levels or ratios, blood pH level, physical and/or psychological stress levels and/or stress level indicators, drug dosage and/or dosimetry, physiological drug reactions, drug chemistry, biochemistry, position and/or balance, body strain, neurological functioning, brain activity, brain waves, blood pressure, cranial pressure, hydration level, auscultatory information, auscultatory signals associated with pregnancy, physiological response to infection, skin and/or core body temperature, facial emotions, eye muscle movement, body movement, geolocation, blood volume, inhaled and/or exhaled breath volume, physical exertion, exhaled breath physical and/or chemical composition, the presence and/or identity and/or concentration of viruses and/or bacteria, foreign matter in the body, internal toxins, heavy metals in the body, anxiety, fertility, ovulation, sex hormones, psychological mood, sleep patterns, hunger and/or thirst, hormone type and/or concentration, cholesterol, lipids, blood panel, bone density, organ and/or body weight, reflex response, electromyography (EMG) signals, electroencephalography (EEG) signals, sexual arousal, mental and/or physical alertness, sleepiness, auscultatory information, response to external stimuli, swallowing volume, swallowing rate, sickness, voice characteristics, voice tone, voice pitch, voice volume, vital signs, head position or tilt, allergic reactions, inflammation response, auto-immune response, mutagenic response, DNA, proteins, protein levels in the blood, water content of the blood, pheromones, internal body sounds, digestive system functioning, cellular regeneration response, healing response, stem cell regeneration response, skin microbiome, gut microbiome, functional near-infrared spectroscopy signals, snoring, satiety, oral microbiome, salivary cortisol and amylase, sweat composition and/or other physiological information.

A physiological sensor may include an impedance plethysmograph for measuring changes in volume within an organ or body (usually resulting from fluctuations in the amount of blood or air it contains). For example, the biometric readout device may include an impedance plethysmograph to monitor blood pressure in real-time.

Pulse oximetry is a standard noninvasive technique of estimating blood gas levels. Pulse oximeters typically employ two or more optical wavelengths to estimate the ratio of oxygenated to deoxygenated blood. Similarly, various types of hemoglobin, such as methemoglobin and carboxyhemoglobin can be differentiated by measuring and comparing the optical absorption at key red and near-infrared wavelengths. Additional wavelengths can be incorporated and/or replace conventional wavelengths. For example, by adding additional visible and infrared wavelengths, myoglobin, methemoglobin, carboxyhemoglobin, bilirubin, SpCO₂, and blood urea nitrogen (BUN) can be estimated and/or monitored in real-time in addition to the conventional pulse oximetry SpO₂ measurement.

Blood hydration can also be monitored optically, as water selectively absorbs optical wavelengths in the mid-IR and blue-UV ranges, whereas water can be more transparent to the blue-green wavelengths. Thus, the same optical emitter/detector configuration used in pulse oximetry can be employed for hydration monitoring. However, mid-IR or blue optical emitters and detectors may be required. Additionally, monitoring the ratio of blue-green to other transmitted or reflected wavelengths may aid the real-time assessment of blood hydration levels. Blood hydration can also be monitored by measuring changes in capacitance, resistance, or inductance in response to varying water-content in the skin tissues or blood. Similarly, hydration can be estimated by monitoring ions extracted via iontophoresis across the skin. Additionally, measuring the return velocity of reflected sound (including ultrasound) entering the head can be used to gauge hydration. These hydration sensors can be mounted anywhere within or along biometric readout device. It should be noted that other hydration sensors can also be incorporated.

A variety of techniques can be used for monitoring blood metabolites. For example, glucose can be monitored via iontophoresis at the surface of the skin combined with enzyme detection. Blood urea nitrogen (BUN) can be monitored by monitoring UV fluorescence in blood (through the skin) or by monitoring visible and mid-IR light absorption using the pulse oximetry approach described above. Various ions such as sodium, potassium, magnesium, calcium, iron, copper, nickel, and other metal ions, can be monitored via selective electrodes following iontophoresis through the skin.

Cardiopulmonary functioning can be evaluated by monitoring blood pressure, pulse, cardiac output, and blood gas levels. Pulse rate and intensity can be monitored through pulse oximetry (described above) as well as by sensing an increase in oxygenated blood with time. Pulse rate and blood flow may also be evaluated through impedance measurements via galvanometry near a blood vessel. Additionally, pulse rate and blood flow may be evaluated through a fast-response thermal energy sensor, such as a pyroelectric sensor. Because moving blood may temporarily increase or decrease the localized temperature near a blood vessel, a pyroelectric sensor will generate an electrical signal that is proportional to the total blood flow in time.

Blood pressure can also be monitored. According to some embodiments of the present invention, a digital blood pressure meter is integrated into the biometric feedback device. A compact clip containing actuators and sonic and pressure transducers can be placed on the skin, and systolic and diastolic pressure can be measured by monitoring the pressure at which the well-known Korotkoff sound is first heard (systolic), then disappears (diastolic). This technique can also be used to monitor intra-cranial pressure and other internal pressures. Blood pressure may also be measured by comparing the time between pulses at different regions of the body.

Electrodes can also be utilized to monitor blood gases diffused through the skin, giving an indication of blood gas metabolism. For example, a compact Severinghaus electrode can be used for the real-time monitoring of CO₂ levels in the blood. These Severinghaus-type electrodes can also be used to monitor other blood gases besides CO₂, such as oxygen and nitrogen.

Organ function monitoring includes monitoring, for example, the liver, kidneys, pancreas, skin, and other vital or important organs. Liver quality can be monitored noninvasively by monitoring optical absorption and reflection at various optical wavelengths. For example, optical reflection from white LEDs or selected visible-wavelength LEDs can be used to monitor bilirubin levels in the skin and blood, for a real-time assessment of liver health.

Monitoring neurological functioning can be accomplished via electrodes. When such electrodes are placed along the forehead, this process is described as electroencephalography, and the resulting data is called an electroencephalogram (EEG). These electrodes can be either integrated into or connected to the biometric feedback device. For example, an earlobe clip can be modified to conform with EEG electrodes or other electrodes for measuring brain waves or neurological activity. For monitoring neurological functioning, a temple earpiece may also be used. Electrodes may be positioned in a temple earpiece region near the temples of a user for direct contact with the skin. In some embodiments, direct contact is not necessary, and the neurological functioning can be monitored capacitively, inductively, electromagnetically, or a combination of these approaches. In some embodiments, brain waves may couple with low frequency acoustical sensors integrated into an earpiece module.

A person's body motion and head position can be monitored by integrating a motion sensor into the biometric feedback device. Two such compact motion sensors include gyroscopes and accelerometers, typically mechanical or optical in origin. In some embodiments, an accelerometer may be composed of one or more microelectromechanical systems (MEMS) devices. In some embodiments, an accelerometer can measure acceleration or position in two or more axes. When the head is moved, a motion sensor detects the displaced motion from the origin.

The number of eye blinks performed over a certain period of time constitutes the so-called spontaneous blink rate (SBR). A contact lens sensor (e.g., Triggerfish; Sensimed AG, Lausanne, Switzerland) can be used to measure changes in ocular circumference and corneal curvature at the corneoscleral junction secondary to changes in intraocular pressure. Measurements from the CLS are obtained in electronic units of voltage (mV) via dilatation of the strain gauge (Gisler, et al. (2015) Transl. Vis. Sci. Technol. 4(1):4). Alternatively, a light emitter/detector device (e.g., using infrared light) can be used to monitor eye movement and blinking. See, e.g., U.S. Pat. No. 6,542,081.

Body temperature, including core and skin temperature, can be monitored in real-time by integrating compact infrared sensors into the biometric feedback device. Infrared sensors are generally composed of thermoelectric/pyroelectric materials or semiconductor devices, such as photodiodes or photoconductors. Thermistors, thermocouples, and other temperature-dependent transducers can also be incorporated for monitoring body temperature. These sensors can be very compact and thus can be readily integrated into the biometric feedback device.

Breathing characteristics can be monitored via auscultatory signal extraction. In some embodiments, an acoustic sensor is used to sense sounds associated with breathing. Signal processing algorithms are then used to extract breathing sounds from other sounds and noise. This information is processed into a breathing monitor, capable of monitoring, for example, the intensity, volume, and speed of breathing. Another method of monitoring breathing is to employ pressure transducers. Changes in pressure inside or near the ear associated with breathing can be measured directly and, through signal processing, translated into a breathing monitor. Similarly, optical reflection sensors can be used to monitor pressure by monitoring physical changes in the skin or tissues in response to breathing. For monitoring the physical changes of the tympanic membrane in response to breathing, and hence ascertaining breathing rate, an optical signal extraction approach may be employed. At least one color sensor, or colorimetric sensor, can be employed to monitor changes in color associated with breathing and other health factors.

Caloric intake, physical activity, and metabolism can be monitored using a core temperature sensor, an accelerometer, a sound extraction methodology, a pulse oximeter, a hydration sensor, and the like. These sensors can be used individually or in unison to assess overall caloric metabolism and physical activity. For example, a sound extraction methodology can be used to extract sounds associated with swallowing, and this can give an indication of total food volume consumed. Additionally, a core temperature sensor, such as a thermopile, a pyroelectric sensor, a thermoelectric sensor, or a thermistor, or a tympanic membrane extraction technique, can be used to assess metabolism. In one case, the core temperature is compared with the outdoor temperature, and an estimate of the heat loss from the body is made, which is related to metabolism.

In addition, skin conductance can be measured using electrodes; facial emotions can be measured using electrodes (electromyography) or a simple facial camera (e.g., using Ekman and Friesen's Facial Action Coding System; see Ekman, et al. (1980) J. Personal. Social Psychol. 39:1125-34); body strain can be measured using strain gauges or electrodes; eye movements/blinks/pupil dilation can be tracked using infrared sensors (e.g., Tobii Pro Glasses; Tobii Technology, Inc., Falls Church, Va.); DNA based biosensors can be used to analyze chemicals in exhaled breath (see, e.g., Ping, et al. (2016) ACS Nano 10(9):8700-8704); and voice analysis can be done use a simple microphone (e.g., Emotions Analytics; Beyond Verbal Communications, LTD, Tel Aviv, Israel).

Turning to the environmental sensor module 30 of the present system, such sensors are configured to detect and/or measure one or more of the following types of environmental information: climate, sound, humidity, temperature, pressure, barometric pressure, soot density, airborne particle density, airborne particle size, airborne particle shape, airborne particle identity, volatile organic compound (VOCs), hydrocarbons, polycyclic aromatic hydrocarbons (PAHs), carcinogens, toxins, electromagnetic energy, optical radiation, X-rays, gamma rays, microwave radiation, terahertz radiation, ultraviolet radiation, infrared radiation, radio waves, atomic energy alpha particles, atomic energy beta-particles, gravity, light intensity, light frequency, light flicker, light phase, ozone, carbon monoxide, carbon dioxide, nitrous oxide, sulfides, airborne pollution, foreign material in the air, viruses, bacteria, signatures from chemical weapons, wind, air turbulence, sound and/or acoustical energy, ultrasonic energy, noise pollution, human voices, animal sounds, diseases expelled from others, exhaled breath and/or breath constituents of others, toxins from others, pheromones from others, industrial and/or transportation sounds, allergens, animal hair, pollen, exhaust from engines, vapors and/or fumes, fuel, signatures for mineral deposits and/or oil deposits, snow, rain, thermal energy, hot surfaces, hot gases, solar energy, hail, ice, vibrations, traffic, the number of people in a vicinity of the person, coughing and/or sneezing sounds from people in the vicinity of the person, loudness and/or pitch from those speaking in the vicinity of the person, and/or other environmental information.

Environmental temperature can be monitored, for example, by thermistor, thermocouple, diode junction drop reference, or the like. Electrical temperature measurement techniques are well-known to those skilled in the art, and are of suitable size and power consumption that they can be integrated into an environmental sensor module without significant impact on the size or functionality of the wireless earpiece module.

Environmental noise can be monitored, for example, by a transducer, microphone, or the like. Monitoring of environmental noise preferably includes, but is not limited to, instantaneous intensity, spectral frequency, repetition frequency, peak intensity, commonly in units of decibels, and cumulative noise level exposures, commonly in units of decibel-hours. This environmental noise may or may not include noise generated by a person wearing the environmental sensor module. Sound made by a person wearing the environmental sensor module may be filtered out, for example, using analog or digital noise cancellation techniques, by directional microphone head shaping, or the like. The environmental noise sensor may or may not be the same sensor as that used for the intended purpose of wireless communication. In some embodiments, the environmental noise sensor is a separate sensor having broader audible detection range of noise level and frequency, at the possible sacrifice of audio quality.

Environmental smog includes VOC's, formaldehyde, alkenes, nitric oxide, PAH's, sulfur dioxide, carbon monoxide, olefins, aromatic compounds, xylene compounds, and the like. Monitoring of one or more of the aforementioned smog components can be performed the using the environmental sensor module of the present invention. Photoionization detectors (PID's) may be used to provide continuous monitoring and instantaneous readings. Other methods of detecting smog components according to embodiments of the present invention include, but are not limited to, electrocatalytic, photocatalytic, photoelectrocatalytic, calorimetric, spectroscopic or chemical reaction methods. Examples of monitoring techniques using the aforementioned methods may include, but are not limited to, IR laser absorption spectroscopy, difference frequency generation laser spectroscopy, porous silicon optical microcavities, surface plasmon resonance, absorptive polymers, absorptive dielectrics, and calorimetric sensors. For example, absorptive polymer capacitors inductors, or other absorptive polymer-based electronics can be incorporated into the environmental sensor module of the present invention. These polymers change size or electrical or optical properties in response to analyte(s) from the environment (such as those described above). The electrical signal from these absorptive polymer electronic sensors can be correlated with the type and intensity of environmental analyte. Other techniques or combinations of techniques may also be employed to monitor smog components. For example, a smog component may be monitored in addition to a reference, such as oxygen, nitrogen, hydrogen, or the like. Simultaneous monitoring of smog components with a reference analyte of known concentration allows for calibration of the estimated concentration of the smog component with respect to the reference analyte within the vicinity of an earpiece user.

In some embodiments of the present invention, environmental air particles can be monitored with a flow cell and a particle counter, particle sizer, particle identifier, or other particulate matter sensor incorporated as part of, or attached to, the environmental sensor module. Non-limiting examples of particles include oil, metal shavings, dust, smoke, ash, mold, or other biological contaminates such as pollen. In some embodiments of the present invention, a sensor for monitoring particle size and concentration is an optical particle counter. A light source is used (e.g., a laser or a laser diode), to illuminate a stream of air flow. However, a directional LED beam, generated by a resonant cavity LED (RCLED), a specially lensed LED, or an intense LED point source, can also be used for particle detection. The optical detector, which is off-axis from the light beam, measures the amount of light scattered from a single particle by refraction and diffraction. Both the size and the number of particles can be measured at the same time. The size of the monitored particle is estimated by the intensity of the scattered light. Additionally, particles can be detected by ionization detection, as with a commercial ionization smoke detector. In this case, a low-level nuclear radiation source, such as americium-241, may be used to ionize particles in the air between two electrodes, and the total ionized charge is detected between the electrodes. As a further example, piezoelectric crystals and piezoelectric resonator devices can be used to monitor particles in that particles reaching the piezoelectric surface change the mass and hence frequency of electromechanical resonance, and this can be correlated with particle mass. If the resonators are coated with selective coatings, certain types of particles can attach preferentially to the resonator, facilitating the identification of certain types of particles in the air near a person wearing an earpiece module. In some embodiments, these resonators are solid state electrical devices, such as MEMS devices, thin film bulk acoustic resonators (FBARs), surface-acoustic wave (SAW) devices, or the like. These compact solid state components may be arrayed, each arrayed element having a different selective coating, for monitoring various types of particles.

In some embodiments of the present invention, environmental air pressure or barometric pressure can be monitored by a barometer. Non-limiting examples of barometric pressure measurement include hydrostatic columns using mercury, water, or the like, foil-based or semiconductor-based strain gauge, pressure transducers, or the like. In some embodiments, semiconductor-based strain gauges are utilized. A strain gauge may use a piezoresistive material that gives an electrical response that is indicative of the amount of deflection or strain due to atmospheric pressure. Atmospheric pressure shows a diurnal cycle caused by global atmospheric tides. Environmental atmospheric pressure is of interest for prediction of weather and climate changes. Environmental pressure may also be used in conjunction with other sensing elements, such as temperature and humidity to calculate other environmental factors, such as dew point. Air pressure can also be measured by a compact MEMS device composed of a microscale diaphragm, where the diaphragm is displaced under differential pressure and this strain is monitored by the piezoelectric or piezoresistive effect.

In further embodiments, environmental humidity, relative humidity, and dew point can be monitored by measuring capacitance, resistivity or thermal conductivity of materials exposed to the air, or by spectroscopy changes in the air itself. Resistive humidity sensors measure the change in electrical impedance of a hygroscopic medium such as a conductive polymer, salt, or treated substrate. Capacitive humidity sensors utilize incremental change in the dielectric constant of a dielectric, which is nearly directly proportional to the relative humidity of the surrounding environment. Thermal humidity sensors measure the absolute humidity by quantifying the difference between the thermal conductivity of dry air and that of air containing water vapor. Humidity data can be stored along with pressure monitor data, and a simple algorithm can be used to extrapolate the dew point. In some embodiments of the present invention, monitoring humidity is performed via spectroscopy. The absorption of light by water molecules in air is well known to those skilled in the art. The amount of absorption at known wavelengths is indicative of the humidity or relative humidity. Humidity may be monitored with a spectroscopic method that is compatible with the smog monitoring spectroscopic method described above.

In addition, environmental parameters, such as, climate, traffic, population density, and air pollutants levels, can be obtained from existing data sources/satellite images; light spectrum can be measured using a spectrometer; and the brightness can be determined through a photometer.

Data from the biometric readout device 20 and environmental sensor module 30 is sent to digital controller 40, which analyzes the data and provides information to scent delivery device 60 and optionally one or more sensory delivery devices 70 to provide a scent-based multisensory experience to a subject 80. The digital controller 40 can be in the same housing as the biometric readout device 20 and/or environmental sensor module 30. By way of illustration, the controller digital could be housed in a digital scent device in the form of athlete's head gear, or the sensory delivery device in the form of a smart home. Alternatively, the digital controller 40 can be in a housing separate from the scent delivery device 60 and sensory delivery device(s) 70. When in a separate house, the digital controller can be located within the vicinity of the subject or be in a remote location. The digital controller can take the form of a “portable electronic device” or “PED” and/or a computer. The phrase “portable electronic device” or “PED” as used herein means a personal digital assistant (PDA), portable television, portable cassette player, portable compact disc (CD) player, portable digital versatile disc (DVD) player, portable radio, laptop or hand-held computer, hand-held electronic game device, smart home device (e.g., AMAZON ECHO), mobile or wireless telephone, and the like. The digital controller can also include an on-board or in-vehicle computer present in a car or truck console.

The digital controller 40 is configured to process signals/data provided by the sensors of the biometric readout device 20, environmental sensor module 30 and/or other data platforms 50. Such other data platforms can include, but are not limited to, meta data (e.g., a personal or work calendar), weather data (e.g., historical weather patterns or weather advisories), traffic data (e.g., historical traffic patterns or traffic advisories), public transportation data, data from car services such as Uber or Lyft, entertainment schedules (e.g., television programs), and demographic data, as well as user preference data and usage history data.

In some embodiments, a digital controller is configured to process signals produced by the physiological and environmental sensors into signals that can be heard and/or viewed by the person being monitored. In some embodiments, the digital controller is configured to selectively extract environmental effects from signals produced by a physiological sensor and/or selectively extract physiological effects from signals produced by an environmental sensor.

In addition to providing feedback to the scent delivery device, information from the physiological and environmental monitoring devices may be used to support a clinical trial and/or study, marketing study, dieting plan, health study, wellness plan and/or study, sickness and/or disease study, environmental exposure study, weather study, traffic study, behavioral and/or psychosocial study, genetic study, a health and/or wellness advisory, and an environmental advisory. The monitoring devices may be used to support targeted advertisements, links, searches or the like through traditional media, the internet, or other communication networks. The monitoring devices may be integrated into a form of entertainments, such as health and wellness competitions, sports, or games based on health and/or environmental information associated with a user.

The scent delivery device 60 can be in the same housing as the biometric readout device 20, environmental sensor module 30 and/or digital controller 40, or be in a separate housing. Ideally, the scent delivery device 60 includes a portable housing which is either a portable electronic device which is used in close proximity to the nose of the user, or is a housing adapted to be worn by a user in close proximity to the nose of the user; and a means for selectively generating scent housed in said housing, wherein the scent travels by diffusion (active and/or passive diffusion) to the user's nose. The term scent as used in the specification and claims means the effluent that is perceived by the olfactory organs.

The phrase “housing adapted to be worn by the user” as used herein means a hat, headset, shoulder harness or neck harness, athletic gear, fashion accessory, smart clothing or jewelry, which is worn by the user; or adhesive or magnet support which is affixed to the skin of the user or wearable digital skin, thereby allowing the scent generating means to be placed in close proximity to or in the user's nose. With reference to jewelry, it is contemplated that the jewelry could adorn the nose, for example as a nose ring or stud that attaches to or pierces the nose. Jewelry that overlays the nose in a hidden, embedded, subtle or bold way could also be used. Similarly, jewelry that attaches, pierces or overlays the face or other parts of the head or upper body is also envisioned. The phrase “close proximity to the user's nose” means about 90 inches, 80 inches, 70 inches, 60 inches, 50 inches, 40 inches, or preferably 30 inches or less (75 cm or less), which is an acceptable distance to allow the scent to reach the nose of the user by diffusion.

Diffusion is a recognized natural phenomenon of the spreading or scattering of material. In the present invention, diffusion moves the scent from the scent generating component to the nose by the ambient air, or the natural flows of air that surrounds the user and the scent delivery device. Optionally, the flow of scent by diffusion can be assisted by use of a heater or a fan or a micropump. The fan employed in the present invention is small and is not intended to cool the user but to provide a current or direction to the air so as to aid in the movement of scent to the nose.

The scent generating component can be small and light so as not to hinder the user. Alternatively, the scent generating component can be a component of a smart home, wherein the delivery device is part of the home's HVAC system. The scent generating component can take on a number of embodiments. For example, in one embodiment, the scent generating component of the present invention includes a support affixed to the housing; one or more scent sources mounted on the support to selectively provide scent to the user's nose; and a release mechanism for selectively releasing scent from the scent sources directly to the user's nose. In some embodiments, the support is a silicon chip, disk, or thin plastic film, one side of which is affixed to the housing, the other side of which allows for scent to be released.

In this first embodiment of the scent generating component, the release mechanism for selectively releasing scent to the user's nose acts on the scent source to release the scent. The release mechanism includes a micro-mechanical system (MEMS), tape or other means, to release the desired scent to or in the nares. The release mechanism can be activated manually by the electronics of the PED or by its own electronics.

The scent source can be of many types for this first embodiment. The scent source can be a micro-container, microcapsule or cavity which contains scent molecules in a liquid or gel form. In this embodiment, the scent source holding the scent molecules is normally, closed, however, when the release mechanism is activated, the scent source is selectively opened to allow the scent molecules to diffuse into the nares towards the olfactory nerve receptors.

The scent source can also be scent molecules which are microencapsulated in heat-sensitive capsules. Under conditions of normal environmental temperatures, the microcapsules remain intact and the scent molecules are contained within. They cannot be sensed by the olfactory receptors. However, the release mechanism selectively heats the microcapsules so that the desired scent source is heated and a certain portion of the scent molecules are liberated and allowed to diffuse to the olfactory receptors. As soon as the microcapsules cool, no more scent molecules are liberated from the microcapsules.

In a second embodiment of the scent generating component, one or more scent sources are mounted on a delivery component housed in said housing and the delivery component selectively delivers scent from the scent sources directly to the user's nose. In accordance with this embodiment, the scent sources are placed near or adjacent to the nares one at a time, or more than one at a time. The delivery component moves the scent source to the user's nose. The scent sources in this second embodiment are the same as those for the first embodiment.

In this second embodiment, the scent source holding the scent molecules is normally closed, however, when it is moved into position adjacent to the nares, it is selectively opened to allow the scent molecules to diffuse into the nares towards the olfactory nerve receptors. Where microencapsulated scent molecules are used, these molecules are moved under the nose and then heated or activated to release the scent. The delivery component in accordance with this embodiment of the invention can be a disk or endless belt rotatably mounted in the house, wherein scent containers are mounted on the disk or belt; one or more tubes or capillary tubes which are bundled together and attached to said housing, wherein one end of the tubes is placed in communication with the scent sources; or a matrix in said housing in which each of said scent containers are held. See U.S. Pat. No. 7,437,061, incorporated herein by reference in its entirety, for various configurations of scent delivery components. In any embodiment, a fan or heater can be employed to assist diffusion and provide a current of air on which the scent molecules travel to the nose.

For digital control of the scent, a microprocessor may be attached by wires to a heater. The microprocessor can be controlled by the electronics in the digital controller or by a separate device (e.g., the biometric readout device or environmental sensor module), which communicates in a conventional way to the microprocessor to control the scent that is released. A heater not only causes the release of scent from scent source or microcapsule, but can also cause an air current by the fact that the air is heated to above ambient temperatures, thereby causing an upward flow of air.

Instead of a heater to activate release of scent, a mechanism can be employed to open and close caps or lids of the scent sources. Specifically, each of the scent sources can be capped with a micromechanical cap, a microelectrical cap, or a molecular cap. These different types of caps are made in a conventional manner and operate in a conventional way to open and close the scent source, thereby controlling the release of scent. A heater can still be employed to promote movement of the scent molecules and provide a current of air to carry the scent to the user's nose. In accordance with this embodiment, a microprocessor is used to control the opening and closing of the caps.

In certain embodiments of this invention, the support is a silicon chip into which capillary tubes and a plurality of microcapsules or cavities (i.e., scent sources) have been etched into the chip. The tubes, often referred to as nanochannels, are typically on the order of a few microns (micrometers) in diameter. They are able to transport scent molecules because the scent molecules are smaller than the diameter of the nanochannels. Each of the plurality of microcapsules or cavities contains a small quantity of a concentrated scent-producing substance and may have a cap to prevent unintended release of the scent. Alternatively, the scent-producing substance may be a solid. Preferably, the microcapsules or cavities are arranged in a matrix grid on the microchip such that a grid of electrodes can be overlaid on or electrically connected to the microcapsules or cavities and connected by wires or other conductors to the microprocessor. In some embodiments, the microprocessor is housed on the microchip.

In use, the microprocessor energizes the proper horizontal and vertical electrodes for the microcapsule or cavity containing the selected scent. A heating element heats up the specific microcapsule located at the intersection of the electrodes to release the scent. Alternatively, a catalyst or other chemical could be released or electrically activated to generate the desired scent. Alternatively, a piezoelectric cap may be positioned over each scent cavity, the cap opening when electrically energized to release the scent. It will be recognized that more than one microcapsule or cavity can be opened at one time thereby allowing for the synthesis of scent by the device itself.

As further examples, the scent delivery device can encompass e-spray technology such as the e-spray olfactometer disclosed in PCT/US2017/018270); a surface acoustic wave (SAW) atomizer (see, e.g., U.S. Pat. No. 8,480,010 or U.S. Pat. No. 5,996,903) or ultrasonic vibrations (see, e.g., WO 2007/026872) to disperse a scent.

As used herein, the term “processor” or “microprocessor” refers to a device that takes one form of information and converts this information into another form, typically having more usefulness than the original form. For example, in this invention, a processor (e.g., of the digital controller) may collect raw physiological or environmental data from various sensors and process this data into a meaningful assessment such as pulse rate, blood pressure, or air quality and, based upon this assessment, direct the scent delivery device to release one or more scents. The connection and programming for the communication between the scent delivery device, environmental sensor module, biometric readout device, one or more sensory delivery devices and digital controller are done in a conventional manner using conventional electronics such as wired (e.g., USB, Ethernet, coax etc.) and wireless (e.g., BLUETOOTH, Infrared, wireless, radio transmitter) approaches.

The battery for the scent delivery device of the present invention can be made internal or external to the device depending on whether the device is to be worn, e.g., on the belt or in a pocket of the user, or merely used in close proximity to the nose of the user, e.g., as with a mobile phone.

With respect to a mobile phone, the present invention can be defined as an improved mobile phone wherein one end of the phone has a microphone and the other end of the phone has a speaker, the improvement being a scent generating mechanism housed in said one end of said mobile phone for selectively providing scent to a user's nose by means of diffusion. Because the scent generating mechanism is housed at the microphone end of the phone, the scent generating mechanism is positioned at or near, in close proximity to the user's nose when the user talks on the phone.

According to some embodiments of the present invention, a method of modulating a subject's physiological state includes receiving physiological and/or environmental information from a subject via portable monitoring devices associated with the subject, and analyzing and optionally storing the received information to identify the physiological and/or environmental status associated with the subject and provide feedback to the scent delivery device so that a scent is delivered to modulate the subject's physiological state. Each monitoring device has at least one physiological sensor and/or environmental sensor. Each physiological sensor is configured to detect and/or measure physiological information from the subject, and each environmental sensor is configured to detect and/or measure environmental conditions in a vicinity of the subject. The physiological information and/or environmental information may be analyzed locally via the monitoring device or may be transmitted to a location geographically remote from the subject for analysis. The collected information may stored and undergo virtually any type of analysis. In some embodiments, the received information may be analyzed to identify and/or predict, e.g., the sleep state or cognitive state of the subject, to identify and/or predict environmental changes in the vicinity of the subject, and to identify and/or predict psychological and/or physiological stress for the subject.

According to some embodiments of the present invention, corrective action information may be communicated to the scent delivery device to provide a scent that benefits the subject. In addition, corrective action information for the subject may be communicated to the subject and/or a third party.

According to some embodiments, the system of the present invention includes a plurality of portable monitoring devices, each comprising at least one physiological sensor and/or environmental sensor, a plurality of portable communication devices, wherein each communication device is in communication with a respective monitoring device and is configured to transmit data from the monitoring devices to a digital controller, which has a processor configured to analyze data and to identify and/or predict health and/or environmental status associated with the subject. Each physiological sensor is configured to detect and/or measure physiological information from a subject, and each environmental sensor is configured to detect and/or measure environmental conditions in a vicinity of the subject. In particular embodiments, the biometric readout device is configured to be worn by a subject (e.g., attached to a body of a respective subject, etc.) and the environmental sensor module is worn by or in the near vicinity of the subject.

BLUETOOTH®-enabled and/or other personal communication devices, e.g., earpiece or FITBIT devices, may be configured to incorporate physiological and/or environmental sensors, according to some embodiments of the present invention. Such devices are typically lightweight, unobtrusive devices that have become widely accepted socially. Exemplary physiological and environmental sensors that may be incorporated into a BLUETOOTH® or other type of device include, but are not limited to accelerometers, auscultatory sensors, pressure sensors, humidity sensors, color sensors, light intensity sensors, pulse oximetry sensors, pressure sensors, etc.

Sensors of the present invention can produce digital or analog signals. Therefore, the devices of this invention can include a signal processor to provide a means of converting the digital or analog signals from the sensors into data that can be transmitted wirelessly by a transmitter. The signal processor may be composed of, for example, signal conditioners, amplifiers, filters, digital-to-analog and analog-to-digital converters, digital encoders, modulators, mixers, multiplexers, transistors, various switches, microprocessors, or the like. For personal communication, the signal processor can optionally process signals received by a receiver into signals that can be heard or viewed by the user. The received signals may also contain protocol information for linking various telemetric modules together, and this protocol information can also be processed by the signal processor.

The signal processor may utilize one or more compression/decompression algorithms (CODECs) used in digital media for processing data. The transmitter can be composed of a variety of compact electromagnetic transmitters. A standard compact antenna is used in the standard BLUETOOTH® headset protocol, but any kind of electromagnetic antenna suitable for transmitting at human-safe electromagnetic frequencies may be used. The receiver can also be an antenna. In some embodiments, the receiving antenna and the transmitting antenna are physically the same. The receiver/transmitter can be, for example, a non-line-of-sight (NLOS) optical scatter transmission system. These systems typically use short-wave (blue or UV) optical radiation or “solar blind” (deep-UV) radiation in order to promote optical scatter, but infrared wavelengths can also suffice.

In some embodiments, the transmitter/receiver is configured to transmit signals from the signal processor to a remote terminal (e.g., the scent delivery device) following a predetermined time interval. For example, the transmitter may delay transmission until a certain amount of detection time has elapsed, until a certain amount of processing time has elapsed, etc.

The power source of the biometric feedback device and environmental sensor module can be any portable power source capable of fitting inside the housing. According to some embodiments, the power source is a portable rechargeable lithium-polymer or zinc-air battery. Additionally, portable energy-harvesting power sources can be integrated into the housing and can serve as a primary or secondary power source. For example, a solar cell module can be integrated into the housing for collecting and storing solar energy. Additionally, piezoelectric devices or microelectromechanical systems (MEMS) can be used to collect and store energy from body movements, electromagnetic energy, and other forms of energy in the environment or from the user. A thermoelectric or thermovoltaic device can be used to supply some degree of power from thermal energy or temperature gradients. In some embodiments, a cranking or winding mechanism can be used to store mechanical energy for electrical conversion or to convert mechanical energy into electrical energy that can be used immediately or stored for later.

Embodiments of the present invention are not limited to devices that communicate wirelessly. However, in some embodiments of the present invention, devices configured to monitor a subject's physiology and/or environment may be wired to a device that stores, processes, and/or transmits data. In some embodiments, this information may be stored on the biometric readout device, environmental sensor module, digital controller or a scent delivery device.

Information collected from each monitoring device may include information that is personal and private and information that can be made available to the public. As such, data storage, according to some embodiments of the present invention, may include a private portion and a public portion. In the private portion, health and environmental data that is personalized for each subject is stored. In the public portion, anonymous health and environmental data is stored and is accessible by third parties.

The system and method of the invention can be used in virtually any environment where a subject's physiological status and environment can be monitored and modified by scent. For example, the system of the invention can be used at home, at work, while driving a car or truck, while shopping, in a hospital or doctor's office setting, at school, at church, at a restaurant, at the gym, in wellness hotels, sports/team training facilities or at a spa. The system is particularly suitable for a closed or controlled environment such as a building or car.

Odors, scents or aromas have been shown to modulate physiological responses in humans including sleep, alertness, cognition, satiety, anxiety and the like. In some embodiments, a system that deploys scent throughout the sleep cycle in a controlled and targeted manner is provided so as to protect overall sleep as well as target specific sleep stages that can facilitate specific cognitive functions, e.g., memory consolidation. Further, the same feedback loop can be used to contribute positively to the waking process, i.e., arousal from sleep, via a digital scent-based alarm.

The integrated system and method of this invention may determine the phase of sleep for a user and present the sensory stimulus, i.e., scent, during training and/or sleep. The determination of sleep phase by monitoring an individual during sleep may be referred to as sleep staging. Sleep staging may be performed using the traditional Rechtschaffen & Kales rules, which classify sleep into six separate stages: wake, rapid eye movement (REM) sleep, S1 (light sleep), S2 (light sleep), S3 (deep sleep), and S4 (deep sleep). Alternative systems for sleep staging have been described and are known to those skilled in the art. Techniques for monitoring physiological changes associated with different stages of sleep may include electroencephalography (EEG) recordings of brain activity, electrooculagraphy (EGG) of eye movement and ocular muscle contractions, electrocardiography (ECG) of heart beats, as well as heart rate or heart rate entropy, respiratory rate, body temperature, eye or body movements (actigraphy), and other techniques, e.g., as described herein.

In addition to sensing the phase of sleep, including awaking from sleep, the system also delivers a stimulus, in particular one or more scents, for the purpose of modulating the phase of a user's sleep/awaking cycle or modulating the quality of sleep and/or the quality or intensity of brain rhythms during a particular phase of sleep. In particular, the scent(s) may mimic or repeat a scent that was intentionally or inadvertently paired with the “learning” of the memory to be modulated.

Physiological changes that occur in response to presentations of scent are used for feedback modulation of the delivered scent. These physiological changes may be monitored during sleep, during awaking, during wakefulness, or during training or testing. The variety of physiological features that can be monitored will be appreciated by one skilled in the art and may include brain rhythms that relate to attention, memory processing, or other cognitive function; heart rate, heart beat entropy, or ECG signals; respiratory rate or entropy; pulse oximetry (blood oxygen content, SpO₂); galvanic skin responses (GSRs); arousal levels; eye gaze; posture; muscle tone; or other physiological signals of interest or appreciated by one skilled in the art.

Desirable feedback in response to identified physiological features may include, but are not limited to, modifying the intensity, modality, or other aspects of scent presentation coupled directly or indirectly to other sensory stimulation modalities for a multisensory consumer experience; modifying the rate, content, or difficulty of training content; delivering a reminder cue to modify attention, gaze, or other aspects of cognition or physiology; or delivering stimuli to affect brain rhythms and/or cognitive processes, including but not limited to increasing the frequency, intensity, or spatial extent of slow wave (delta) rhythms. Moreover, scent may be modulated to recapitulate prior physiological activity measured during past successful sleep sessions. For example, a strong stimulus intensity could negatively affect the stability of the current sleep phase, whereas a weak stimulus intensity may not be sufficiently salient to warrant memory consolidation. By monitoring the effect on sleep, memory, or other aspects of physiological function, the present invention can choose the appropriate stimulus parameters for a particular user given a particular set of physiological measurements.

One variation of the system of the invention is configured to aid or enhance the development of a skill. For example, in some variations, the system may be configured to aid in learning foreign languages or technical software languages. Training sessions may be paired with a scent. In general, the scent is distinct from the material being trained. For example, the scent may be a scent that does not, in the absence of being paired with the training session, evoke the trained material. Thus, the system and method described herein may be particularly useful for repeated learning where it is desirable or necessary to enhance learning of more than one piece of information or task skill.

The system described herein may also be used as part of a therapeutic method to treat a patient. For example, the system and method may be used to improve memory and/or cognitive function by individuals with inherited neurodevelopmental disorders characterized by learning, memory and/or cognitive deficits including Down syndrome, Rett syndrome, fragile X syndrome, neurofibromatosis type 1, tuberous sclerosis, phenylketonuria, maple syrup urine disease, and other inherited neurodevelopmental disorders appreciated by one skilled in the art, as well as disorders such as autism spectrum disorders which are generally diagnosed in the first five years of life and may be due to genetic and/or environmental causes.

In some embodiments, the device and/or related method may be used to improve memory and/or cognitive function by individuals with cognitive and/or memory deficits associated with normal aging or neurodegenerative disorders including Alzheimer's disease, Parkinson's disease, frontotemporal dementia, and other age-related or neurodegenerative disorders appreciated by one skilled in the art.

In other embodiments, the system and method may be used by individuals with disorders of sleep such as central sleep apnea, obstructive sleep apnea, insomnia, and other forms of sleep abnormalities appreciated by one skilled in the art, including but not limited to those that lead to reduced memory function or cognitive impairment.

In further embodiments, the system and method may be used by individuals with disorders for which memory disruption is desired such as post-traumatic stress disorder (PTSD), obsessive compulsive disorder, depression, or other disorders appreciated by one skilled in the art.

The instant system and method may be used by adults and/or children. For example, the system and/or related method described herein may be adapted for use by babies, toddlers, or pre-kindergarten-aged children. In this application, one embodiment is a system built into a toy that a child may interact with and/or a piece of clothing intended for the child to wear to bed.

The scent-based system coupled to other sensory modalities may also be used simply to facilitate falling asleep and/or awakening from sleep under optimal ambient conditions as dictated by the end users bespoke physiological readout.

Scents, fragrances, odors or scents of use in this invention and the associated physiological response thereto include, but are not limited to, those listed in Table 1.

TABLE 1

| Scent | Response | Reference |
|---|---|---|
| Olive oil aroma, | Satiety | Frank, et al. |
| specifically hexanal and | | (2013) Am. J. Clin. |
| 2E-hexenal | | Nutr. 98: 1360-6 |
| “Neutral” sweet smells, | Satiety | Hirsch & Gomez |
| including banana, green | | (1995) J. Neurol. |
| apple, vanilla, and | | Med. Surg. 16: 28-31 |
| peppermint | | |
| Rose oil, sandalwood oil, | Sleep | US20120052139 |
| neroli oil and ylang-ylang | induction | |
| oil | | |
| Vetiver | Sleep | US20120272958 |
| | arousal | |
| Vanillin | S2 of the | US20120272958 |
| | sleep | |
| | cycle | |
| Chocolate | Stress and | Stone, et al. |
| | mood | (1987) J. Personal. |
| | | Soc. Psychol. |
| | | 52: 988-993 |
| BANGALOL ™, basil oil, cis- | Relaxation | US20040063604 |
| hex-3-enol, coumarin, | | |
| ethylene brassylate, ethyl | | |
| linalol, FLOROSA ™, | | |
| GALAXOLIDE ™, geraniol, | | |
| cyclohexadecanolide, | | |
| cyclopentadecanone, methyl | | |
| anthranilate, alpha-iso- | | |
| methyl ionone, PRUNELLA ™, | | |
| SILVANONE ™, alpha- | | |
| terpineol, TRASEOLIDE ™, | | |
| ULTRAVANIL ™, gamma- | | |
| undecalactone, vetiver | | |
| oil, or vetiver acetate | | |
| Clary Sage, Tangerine, | Relaxation | US20040244793 |
| Lavender, and Jojoba | | |
| Orange, Peppermint, | Alertness | US20040244793 |
| Eucalyptus, Lemongrass, | | |
| and Jojoba | | |
| Lemon | Nausea | Yavari, et al. |
| | | (2014) Iran Red |
| | | Cresent Med. J. |
| | | 16: e14360 |

A digital scent-based feedback loop between controlled scent stimulation and biometric readouts can also be integrated with other sensory inputs including but not restricted to visual, auditory and haptic, for a multisensory experience. The scent-based digital feedback loop described herein will provide consumers the necessary control and information to reinforce desired behavioral, cognitive and habit changes, in a self-driven and proactive manner that can lead to individual peak productivity/performance.

In addition to the above-described embodiments, the instant system could also provide a “semi-closed-loop” functionality. By way of illustration, the system would include a microenapsulated product, e.g., fabric bed sheets and/or pillow case, such that ambient VOCs and biometrics would provide a scent release profile linked to a biometric readout for personal feedback to alert the user when to recharge the sheets. In accordance with such a system, the sensors and digital display could be fully integrated via the fabric.

The invention is described in greater detail by the following non-limiting examples.

### EXAMPLE 1

### Sleep Modulation

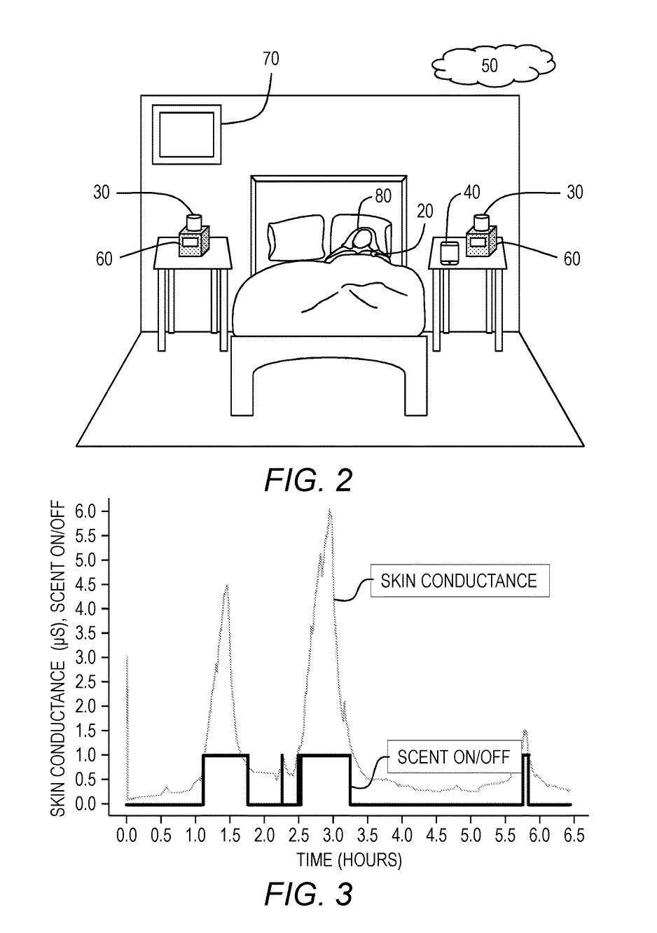

A sleep-based system is designed to accelerate sleep onset, maintain optimal sleep pattern and ensure refreshed awakening. As illustrated in FIG. 2, the sleep-based system includes a biometric readout device 20, environmental sensor modules 30, and a digital controller 40 for receiving data from the biometric readout device 20 and environmental sensor modules 30, as well as data from other data platforms 50, and sending signals to a scent display device 60 (illustrated as sharing housing with the environmental sensor modules 30) and sensory delivery device(s) 70. Ideally, the biometric readout device includes a pulse oximeter, galvanometer or pyroelectric sensor for monitoring heart rate; an infrared sensor, thermistor or thermocouple for monitoring skin temperature; a skin conductance sensor or algesimeter or galvactivator for monitoring skin conductance; an acoustic sensor, pressure transducer or optical reflection sensor for monitoring respiration rate; a gyroscope or accelerometer for monitoring movement; and Wi-Fi-based positioning, global positioning or indoor positioning for monitoring geolocation. In addition, the system includes environmental sensor modules 30 having a thermistor or thermocouple for monitoring temperature; an optical detector for monitoring light; a spectrometer or resistive, thermal or capacitive humidity sensor for monitoring humidity; and a photoionization detector for monitoring VOCs. The digital controller 40 of the system receives data from the biometric readout device 20 and environmental sensor modules 30, as well as data from other data platforms 50 and sends signals to a scent display device and sensory delivery devices such as a lighting system, HVAC, and humidifier/dehumidifier.

In the preferred embodiment, the sensors of the biometric readout device include an accelerometer, EEG, heart-rate sensor, skin conductance sensor, respiration monitor, and skin temperature sensor housed in a wearable device. In a further preferred embodiment, the system includes environmental sensor modules for monitoring sound, light (brightness and spectrum), temperature, humidity, and VOC. In yet a further embodiment, the system includes one or more other data platforms. In one embodiment, the environmental sensor module(s), sensory delivery device, and digital controller are in separate housings. In another embodiment, the sensory delivery device is portable. In a further embodiment, the digital controller is portable. In another embodiment, the environmental sensor module(s), sensory delivery device, and digital controller are in the same housing assembly. In yet a further embodiment, the environmental sensor module(s), scent delivery device, sensory delivery device, and digital controller are in the same housing assembly.

Sleep Onset. The system prepares the bedroom for optimal sleep based on geolocation, time, historical pattern, scent use history, and explicit user preferences by setting the thermostat to optimal temperature (65° F. or 18.5° C.), dimming the lights, releasing the scent designed for increasing relaxation (e.g., one or a combination of clary sage, tangerine, lavender, and jojoba) and/or reducing sleep onset time (e.g., one or a combination of lavender, neroli, lavandin, petitgrain bigarade, jasmine, vetiver, and ylang-ylang).

Optimal Sleep. After the user lays down in bed, the system monitors biometric signals including but not limited to skin conductance, heart rate, skin temperature, movements, respiration rate to determine sleep stages (N1, N2, N3, REM etc.) and algorithmically releases a specific scent to optimize and protect individual sleep stages. The amount of scent released is determined based on real-time monitoring of vapor concentration in the room and user preferences. For example, a skin conductance sensor was used to monitor skin conductance as a measure of stress. When combining the skin conductance sensor with a scent delivery device in a closed-loop manner, stress levels could be modulated during sleep by releasing a scent for increasing relaxation (e.g., one or a combination of clary sage, tangerine, lavender, and jojoba). See FIG. 3.

Refreshed Awakening. The system initiates the wake up process to ensure that the user wakes up optimally refreshed. The wake up process includes, but is not limited to, monitoring user's sleep cycle through biometric measurements, user's preference, user's current (planned physical and mental activities) and previous day's agenda (stress levels, physical and mental activities) to trigger the release of a specific scent at a specific dosage and change lighting, temperature, humidity of the room. For example, depending on where a subject is in the sleep cycle, the system can begin to gently wake the subject 30 minutes before the set wake time by triggering the release of a particular scent for refreshed awakening (e.g., vetiver, orange, peppermint, eucalyptus, lemongrass, basil oil, neroli, peppermint, ginger oil, orange bigarade, and/or petitgrain citronnier).

Power Naps. The three phases of sleep onset, optimal sleep and refreshed awakening can also be optimized for a ˜40 minute power nap, e.g., optimizing light and REM stage sleep as opposed to deep sleep.

### EXAMPLE 2

### Automobile System

An automobile-based system is designed to personalize a traditional or automated driving experience for the driver and/or passenger. The automobile-based system includes a skin conductance sensor or algesimeter or galvactivator for monitoring skin conductance; a biometric readout device including a pulse oximeter, galvanometer or pyroelectric sensor for monitoring heart rate; an infrared sensor, thermistor or thermocouple for monitoring skin temperature; a gyroscope or accelerometer for monitoring movement and/or head position; an acoustic sensor, pressure transducer or optical reflection sensor for monitoring respiration rate; and a contact lens sensor or infrared light emitter/detector for monitoring eye blinks and movements. In addition, the system includes environmental sensor modules such as an optical sensor for detecting ice, rain or snow by means of light absorption; a pressure sensor and accelerometer for assessing slip and friction; a microphone for monitoring tire noise; global positioning for monitoring geolocation; and a thermistor or thermocouple for monitoring temperature. The digital controller of the system receives data from the biometric readout device and environmental sensor modules, as well as data from other data platforms and sends signals to a scent display device and sensory delivery devices such as the car air conditioning system.

In one embodiment, the sensors of the biometric readout device are embedded into the steering wheel of the automobile. In another embodiment, the environmental sensors, digital controller, scent display device and sensory delivery device(s) are housed in the dashboard of the automobile. In a further embodiment, the user can use the automobile's media console or a cell phone app to interface with the system.

Driver. The system is designed for pleasurable and safe car driving experience. The system monitors a driver's biometric signals including but not limited to skin conductance, heart rate, skin temperature, movements, respiration rate, eye blinks, eye movements, and head position to determine the driver's stress level, drowsiness, focus/distraction level, nausea (motion sickness/kinetosis), etc. The system further determines road conditions through sensors on the car, traffic advisories, and real-time traffic maps and uses this information in real-time to trigger specific scent or mixture of scents to alleviate these conditions (e.g., chocolate for stress; lemon for nausea; or orange, peppermint, eucalyptus, or lemongrass for drowsiness). Additionally, the system also takes user preference into consideration to determine the scent identity and scent dosage. The scent delivery device, biometric sensors, car sensors are in a continuous closed feedback loop to ensure the car driving experience is both safe and pleasurable.

Passenger. The system is designed to make car riding experience relaxing and pleasurable. The system monitors a passenger's biometric signals including but not limited to skin conductance, heart rate, skin temperature, movements, respiration rate, eye blinks, eye movements, and head-position to determine riders' stress levels, drowsiness, focus/distraction level, nausea etc. It than takes into account the passenger's mood preferences (relaxing, romantic etc.) to determine the scent displayed and also synchronizes it with the music being played in the car.

### EXAMPLE 3

### System for Modulating Workplace Productivity

The system is designed to make the user more productive in the workplace. The workplace system includes a biometric readout device including a skin conductance sensor or algesimeter or galvactivator for monitoring skin conductance; a pulse oximeter, galvanometer or pyroelectric sensor for monitoring heart rate; an infrared sensor, thermistor or thermocouple for monitoring skin temperature; a an acoustic sensor, pressure transducer or optical reflection sensor for monitoring respiration rate; a gyroscope or accelerometer for monitoring movement; and a contact lens sensor or infrared light emitter/detector for monitoring eye blinks and movements. In addition, the system includes environmental sensor modules such as a thermistor or thermocouple for monitoring temperature; an optical detector for monitoring light; a spectrometer or resistive, thermal or capacitive humidity sensor for monitoring humidity; and a noise dosimeter for monitoring sound levels. The digital controller of the system receives data from the biometric readout device and environmental sensor modules, as well as data from other data platforms and sends signals to a scent display device and sensory delivery devices such as a lighting system, HVAC, humidifier/dehumidifier and sound speaker.

In a preferred embodiment, the sensors of the biometric readout device include an accelerometer, EEG, heart-rate sensor, skin conductance sensor, respiration monitor, and skin temperature sensor that could be housed in a wearable device. In a further preferred embodiment, the system includes environmental sensor modules for monitoring sound, light (brightness and spectrum), and VOC. In yet a further embodiment, the system includes one or more other data platforms. In one embodiment, the environmental sensor module(s), sensory delivery device, digital controller and scent delivery device are in the same housing assembly, e.g., that can reside on the desktop.

The workplace system monitors a user's biometric signals including but not limited to skin conductance, heart rate, skin temperature, movements, respiration rate, eye blinks, eye movements, to determine users' stress levels, drowsiness, focus/distraction level, and mood. The system also takes into account the meta-data from the user's calendar, historical data and the user's preferences. Using the combination of data, the system provides a specific scent or mixture of scents and alters ambient lighting, temperature, humidity and sound levels to bring the user to a state of optimal performance. The scent delivery device, biometric sensors and environmental sensors (temperature, humidity, lighting and sound levels etc.) are in a continuous closed feedback loop to bring the user to a state of optimal performance throughout the day.

### EXAMPLE 4

### Smart Home System

The system is designed to optimize each functional space/room within the home (e.g., entrance, dining, family room, kitchen, living room, bedroom, home office etc.) by triggering scents and altering ambient parameters (lighting, temperature etc.) based on user preferences, meta data, ambient and biometric sensors algorithmically in a closed loop. In certain embodiments, the system is a whole house system, wherein each room/space can be configured independently with the bedroom configured for relaxation, home-office/study configured for focus, entrance configured for welcoming/caring, children's room configured with happy/caring, and family space configured for happiness, relaxation etc.

### EXAMPLE 5

### Wearable, Personal Accessory System

The system is designed to be worn by a subject, e.g., as a necklace, watch or headgear. In one embodiment, the biometric readout device, digital controller and scent delivery device are in the same wearable device. By way of illustration, the wearable system can be used in the form of head gear that provides an athlete with an energy boosting fragrance when the biometric readout device detects fatigue.

What is claimed is:

1. A system comprising
(a) a biometric readout device,
(b) one or more environmental sensor modules, and
(c) a digitally controlled scent delivery device,
wherein the biometric readout device and environmental sensor modules are in a closed feedback loop with the digitally controlled scent delivery device to control scent delivery based on physiological and environmental conditions.

2. The system of claim 1, wherein the biometric readout device comprises at least one physiological sensor configured to detect or measure physiological information from a subject.

3. The system of claim 1, wherein the one or more environmental sensor modules comprise at least one environmental sensor configured to detect or measure environmental conditions in a vicinity of a subject.

4. The system of claim 3, wherein the environmental conditions comprise temperature, sound, humidity, light or volatile organic compounds.

5. The system of claim 1, further comprising one or more other sensory delivery devices.

6. The system of claim 5, wherein the one or more other sensory delivery devices comprise devices for modulating light, sound, temperature, pressure, visual stimulus or haptics.

7. The system of claim 1, further comprising a digital controller.

8. The system of claim 7, wherein the digital controller receives data from the biometric readout device, the one or more environmental sensor modules and other data platforms.

9. A method of modulating a subject's physiological state comprising
(a) receiving physiological and environmental information from a subject via a biometric readout device and one or more environmental sensor modules associated with the subject;
(b) analyzing the received information to identify the physiological and/or environmental status associated with the subject;
(c) providing feedback to a scent delivery device based upon the subject's physiological and/or environmental status; and
(d) delivering a scent from the scent delivery device thereby modulating the subject's physiological state.

10. The method of claim 9, further comprising (e) delivering one or more other sensory modalities to provide a scent-based multisensory experience.

11. The method of claim 10, wherein the one or more other sensory modalities comprise light, sound, temperature, pressure, visual stimulus or haptics.

12. The method of claim 9, wherein the subject's physiological state comprises the subject's sleep cycle.

13. The method of claim 9, wherein the subject's physiological state comprises performance of the subject during a cognitive or motor task.

14. The method of claim 13, wherein the cognitive or motor task comprises driving a car.
